SEQUENCE LISTING (2 sequences)

SEQ ID NO: 1 - DNA, 19 bp, Corynebacterium glutamicum
  misc_feature (1)..(19): Primer tipA-int1
cgcctttacacagaagacg

SEQ ID NO: 2 - DNA, 19 bp, Corynebacterium glutamicum
  misc_feature (1)..(19): Primer tipA-int2
gtgtaccactgaccgatgc

(19) United States
(12) Patent Application Publication
(10) Pub. No.: US 2005/0221454 A1
(43) Pub. Date: Oct. 6, 2005

(54) Process for the production of L-amino acids using coryneform bacteria

(76) Inventor: Brigitte Bathe, Salzkotten (DE)

Correspondence Address: FITCH, EVEN, TABIN & FLANNERY, P. O. BOX 65973, WASHINGTON, DC, 20035, US

(21) Appl. No.: 11/074,025

(22) Filed: Mar. 8, 2005

(30) Foreign Application Priority Data
Mar. 9, 2004 (DE) 10 2004 011 248.7

(51) Int. Cl. C12P 13/08; C12N 15/74; C12N 1/21

(52) U.S. Cl. 435/115; 435/252.3; 435/471

(57) ABSTRACT

The pesent invention relates to a process for the production of L-amino acids, in which the following steps are carried out: a) fermentation of a coryneform bacteria producing the desired L-amino acid, in which bacteria at least the gene coding for the transcription regulator TipA is attenuated, b) concentration of the desired L-amino acid in the medium or in the cells of the bacteria, and c) isolation of the L-amino acid.

### CROSS REFERENCE TO RELATED APPLICATIONS

The present application claims priority to German application 10 2002 011 248.7, filed on Mar. 9, 2004, the content of which is hereby incorporated by reference.

### FIELD OF THE INVENTION

The invention is directed to a process for the production of L-amino acids, especially L-lysine, using coryneform bacteria in which the tipA gene, coding for the transcription regulator TipA, has been attenuated.

### BACKGROUND

L-amino acids are used in human medicine, in the pharmaceutical industry, in the foodstuffs industry and in animal feeds. These amino acids are often produced by the fermentation of strains of coryneform bacteria, especially Corynebacterium glutamicum. Because of their economic importance, work is constantly being carried out to improve the production processes. Improvements may be concerned with fermentation methodology (e.g., the way in which cultures are stirred and oxygenated), the composition of the nutrient medium present during fermentation (e.g., the sugar concentration present), the way in which the product formed is isolated (e.g., by ion-exchange chromatography), or the intrinsic performance properties of the microorganism itself.

In order to improve the performance of amino acid-producing microorganisms, methods of mutagenesis, selection and mutant selection are used. These methods may yield strains that produce L-amino acids such as threonine or lysine and that are either resistant to antimetabolites (such as the threonine analogue α-amino-β-hydroxyvaleric acid (AHV) or the lysine analogue S-(2-aminoethyl)-L-cystein (AEC)), or that are auxotrophic for metabolites of regulatory importance. Methods of recombinant DNA technology have also been employed to improve Corynebacterium glutamicum strains producing L-amino acids.

### DESCRIPTION OF THE INVENTION

The invention is directed to a process for the production of L-amino acids using coryneform bacteria in which at least the nucleotide sequence coding for the transcription regulator TipA is attenuated, especially excluded or expressed at a low level. In addition, the invention provides a process for the production of L-amino acids, in which the following steps are carried out:
a) fermentation of an L-amino-acid-producing coryneform bacteria, in which at least the nucleotide sequence coding for the transcription regulator TipA is attenuated, especially excluded or expressed at a low level;
b) concentration of the L-amino acids in the medium or in the cells of the bacteria; and
c) isolation of the desired L-amino acid, portions or the totality of constituents of the fermentation liquor and/or of the biomass optionally remaining in the end product.

The coryneform bacteria used preferably already produce L-amino acids, especially L-lysine, before attenuation of TipA. As described further herein, it has been found that such attenuation causes the bacteria to produce these amino acids in an improved manner.

Transcription regulators are proteins that bind to DNA by means of a specific protein structure, called the helix-turn-helix motif, and can thus either enhance or attenuate the transcription of other genes. It has been found that TipA represses genes involved in L-amino acid biosyntheses, especially in L-lysine biosynthesis. The nucleotide sequence of the gene coding for TipA of Corynebacterium glutamicum may be found in patent application EP1108790 as sequence no. 2871 and as sequence no. 7068. The nucleotide sequence has also been deposited in the databank of the National Center for Biotechnology Information (NCBI) of the National Library of Medicine (Bethesda, Md., USA) under Accession Number AX122955 and under Accession Number AX127152.

The tipA sequences described in the above references, can be used in accordance with the invention. It is also possible to use alleles of tipA which result from the degeneracy of the genetic code or from function-neutral sense mutations.

The term “L-amino acids” or “amino acids” as used herein means one or more amino acids, including their salts, selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-lysine is particularly preferred. Unless otherwise indicated, the term “L-lysine” or “lysine” as used herein includes not only the amino acid itself but also salts such as lysine monohydrochloride or lysine sulfate.

The term “attenuation” in this context means reducing or eliminating the intracellular activity of one or more enzymes in a microorganism that are coded for by the corresponding DNA. This may be accomplished, for example, using a weak promoter, using a gene or allele that codes for a corresponding enzyme having a low level of activity, or by rendering the corresponding gene or enzyme inactive, and optionally combining these measures. Attenuation will generally result in the activity or concentration of the corresponding protein being lowered to 0 to 75%, 0 to 50%, 0 to 25%, 0 to 10% or 0 to 5% of the activity or concentration of the wild-type protein, or of the activity or concentration of the protein in the starting microorganism.

A reduction in protein concentration can be demonstrated by 1- and 2-dimensional protein gel separation and subsequent optical identification of the protein concentration using corresponding evaluation software. A common method for preparing protein gels in the case of coryneform bacteria and for identifying the proteins is the procedure described by Hermann et al. (Electrophoresis 22:1712-23 (2001)).

Protein concentration can also be analysed by Western blot hybridisation using an antibody specific for the protein (Sambrook et al., Molecular Cloning: A Laboratory Manual, 2^{nd} ed., Cold Spring Harbor Laboratory Press, Cold Spring Harbor, N.Y., 1989) and subsequent optical evaluation using appropriate software for concentration determination (Lohaus, et al., Biospektrum 5:32-39 (1998); Lottspeich, Angewandte Chemie 111:2630-2647 (1999)). The activity of DNA-binding proteins can be measured by means of DNA band-shift assays (also referred to as gel retardation assays, Wilson et al. J. Bacteriol. 183:2151-2155 (2001)). The effect of DNA-binding proteins on the expression of other genes can be demonstrated by various reporter gene assays (Sambrook et al., Molecular Cloning: A Laboratory Manual, 2^{nd} Ed., Cold Spring Harbor Laboratory Press, Cold Spring Harbor, N.Y., 1989).

The microorganisms provided by the present invention are able to produce amino acids from glucose, sucrose, lactose, fructose, maltose, molasses, starch, cellulose or from glycerol and ethanol. They may be coryneform bacteria, especially of the genus Corynebacterium. In the case of the genus Corynebacterium, a particularly preferred species is Corynebacterium glutamicum, which is known to those skilled in the art for its ability to produce L-amino acids. Suitable strains of the genus Corynebacterium, especially of the species Corynebacterium glutamicum, are the wild-type strains:
Corynebacterium glutamicum ATCC 13032;
Corynebacterium acetoglutamicum ATCC15806;
Corynebacterium acetoacidophilum ATCC 13870;
Corynebacterium melassecola ATCC17965;
Corynebacterium thermoaminogenes FERM BP-1539;
Brevibacterium flavum ATCC14067;
Brevibacterium lactofermentum ATCC 13869; and

Brevibacterium divaricatum ATCC 14020,

and L-amino-acid-producing mutants and strains produced therefrom, such as, for example, the L-lysine-producing strains:

Corynebacterium glutamicum FERM-P 1709;
Brevibacterium flavum FERM-P 1708;
Brevibacterium lactofermentum FERM-P 1712;
Corynebacterium glutamicum FERM-P 6463;
Corynebacterium glutamicum FERM-P 6464; and
Corynebacterium glutamicum DSM 5715.

In order to achieve an attenuation, either the expression of the gene coding for TipA or the regulatory properties of the gene product can be reduced or excluded. The two measures may also, optionally, be combined. Gene expression can be diminished by culturing bacteria in a suitable manner or by genetic alteration (mutation) of the signal structures of gene expression. Signal structures of gene expression are, for example, repressor genes, activator genes, operators, promoters, attenuators, ribosome-binding sites, the start codon and terminators. A person skilled in the art will find relevant information concerning this, for example, in patent application WO 96/15246, in Boyd, et al., (J. Bacteriol. 170:5949 (1988)), in Voskuil et al. (Nucl. Ac. Res. 26:3548 (1998), in Jensen, et al., (Biotechnol. Bioeng. 58: 191 (1998)), in Pátek et al. (Microbiology 142: 1297 (1996)) and in textbooks of genetics and molecular biology, such as that of Knippers (Molekulare Genetik, 6th ed., Georg Thieme Verlag, Stuttgart, Germany, 1995) or that of Winnacker (Gene und Klone, VCH Verlagsgesellschaft, Weinheim, Germany, 1990).

A further method of specifically reducing gene expression utilizes antisense technology, in which short oligodeoxynucleotides or vectors are introduced into the target cells for the synthesis of longer antisense RNA. The antisense RNA is able to bind to complementary sections of specific mRNAs and reduce their stability or block their translatability. The person skilled in the art will find an example thereof in Srivastava et al. (Appl. Environ. Microbiol. 66:4366-4371 (2000)).

Mutations that lead to a change in or diminution of the catalytic properties of enzymes are also known from the prior art (see e.g., Qiu, et al., J. Biol. Chem. 272:8611-8617 (1997); Sugimoto et al., Biosci. Biotech. Biochem. 61:1760-1762 (1997) and Möckel, Die Threonindehydratase aus Corynebacterium glutamicum: Aufhebung der allosterischen Regulation und Struktur des Enzyms, Berichte des Forschungszentrums Jülich, Jül-2906, ISSN09442952, Jülich, Germany, 1994). Summaries may also be found in textbooks of genetics and molecular biology, such as that of Hagemann (Allgemeine Genetik, Gustav Fischer Verlag, Stuttgart, 1986).

Mutations may take the form of transitions, transversions, insertions and deletions. Depending on the effect of the amino acid substitution on enzyme activity, the terms missense mutations or nonsense mutations are used. Insertions or deletions of at least one base pair in a gene may lead to frame shift mutations, as a result of which incorrect amino acids are incorporated into proteins or translation breaks off prematurely. Deletions of several codons typically lead to a complete loss of enzyme activity. Instructions for the production of such mutations can be found in textbooks of genetics and molecular biology, such as the textbook of Knippers (Molekulare Genetik, 6th edition, Georg Thieme Verlag, Stuttgart, Germany, 1995), that of Winnacker (Gene und Klone, VCH Verlagsgesellschaft, Weinheim, Germany, 1990) or that of Hagemann (Allgemeine Genetik, Gustav Fischer Verlag, Stuttgart, 1986).

Common methods of mutating genes of C. glutamicum include the methods of gene disruption and gene replacement described by Schwarzer et al. (Bio/Technology 9:84-87 (1991)). In the method of gene disruption, a central portion of the coding region of the gene in question is cloned into a plasmid vector which is capable of replication in a host (typically E. coli) but not in C. glutamicum. Suitable vectors are, for example, pSUP301 (Simon, et al., Bio/Technology 1:784-791 (1983)), pK18mob or pK19mob (Schafer et al., Gene 145:69-73 (1994)), pK18mobsacB or pK19mobsacB (Jager, et al., J. Bacteriol. 174:5462-65 (1992)), pGEM-T (Promega corporation, Madison, Wis., USA), pCR2.1-TOPO (Shuman, J. Biol. Chem 269:32678-84 (1994); U.S. Pat. No. 5,487,993), pCR®Blunt (Invitrogen, Groningen, Netherlands; Bernard, et al., J. Mol. Biol. 234:534-541 (1993)) or pEM1 (Schrumpf, et al., J. Bacteriol. 173:4510-4516 (1991)). The plasmid vector containing the central portion of the coding region of the gene is then transferred to the desired strain of C. glutamicum by conjugation or transformation. The method of conjugation is described, for example, in Schafer, et al. (Appl. Environ. Microbiol. 60:756-759 (1994)). Methods of transformation are described, for example, in Thierbach et al. (Appl. Microbiol. Biotechnol. 29, 356-362 (1988)), Dunican, et al. (Bio/Technol. 7:1067-1070 (1989)) and Tauch, et al. (FEMS Microbiol. Lett. 123, 343-347 (1994)). After homologous recombination by means of a cross-over occurrence, the coding region of the gene in question is disrupted by the vector sequence, and two incomplete alleles lacking the 3′- and the 5′-end, respectively, are obtained. This method has been used, for example, by Fitzpatrick et al. (Appl. Microbiol. Biotechnol. 42:575-580 (1994)) to exclude the recA gene of C. glutamicum.

In the gene replacement method, a mutation, such as a deletion, insertion or base substitution, is produced in vitro in the gene in question. The allele that is produced is, in turn, cloned into a vector that is not replicative for C. glutamicum, and the latter is then transferred to the desired host of C. glutamicum by transformation or conjugation. After homologous recombination by means of a first cross-over occurrence effecting integration and by means of a suitable second cross-over occurrence effecting an excision in the target gene or in the target sequence, incorporation of the mutation or of the allele is achieved. This method has been used, for example, by Peters-Wendisch et al. (Microbiol. 144:915-927 (1998)) to exclude the pyc gene of C. glutamicum by means of a deletion. It is possible in this manner to incorporate a deletion, insertion or base substitution into the gene coding for TipA.

It may also be advantageous for the production of L-amino acids, in addition to attenuating the gene coding for TipA, to enhance, especially overexpress, one or more enzymes of the relevant L-amino acid biosynthesis pathway, of glycolysis, of the anaplerotic pathway, of the citric acid cycle, of the pentose phosphate cycle, of amino acid export, or to enhance one or more regulatory proteins. The term “enhancement” or “enhance” in this context describes increasing the intracellular activity of one or more enzymes or proteins in a microorganism that are coded for by the corresponding DNA, by, for example, increasing the copy number of the gene or genes, using a strong promoter or a gene that codes for a corresponding enzyme or protein having a high level of activity, and optionally combining these measures. Enhancement, especially overexpression, may result in the activity or concentration of the corresponding protein being increased by at least 10%, 25%, 50%, 75%, 100%, 150%, 200%, 300%, 400% or 500%, by a maximum of 1000% or 2000%, relative to that of the wild-type protein or to the activity or concentration of the protein in the starting microorganism. The use of endogenous genes is generally preferred. The expression “endogenous genes” or “endogenous nucleotide sequences” is understood to mean the genes or nucleotide sequences present in the population of a species. Accordingly, for the production of L-lysine, it is possible, in addition to attenuating the gene coding for TipA, to enhance, especially overexpress, one or more genes selected from the group:
the lysC gene coding for a feedback-resistant aspartate kinase (Accession No. P26512, EP-B-0387527; EP-A-0699759; WO 00/63388);
the lysE gene coding for the lysine export protein (DE-A-195 48 222);
the gap gene coding for glyceraldehyde-3-phosphate dehydrogenase (Eikmanns, J. Bacteriol. 174:6076-6086 (1992));
the pyc gene coding for pyruvate carboxylase (EP-A-1083225);
the zwf gene coding for glucose-6-phosphate dehydrogenase (JP-A-09224661; WO 01/70995);
the mqo gene coding for malate:quinone oxidoreductase (Molenaar et al., Eur. J. Biochem. 254:395-403 (1998); EP-A-1038969);
the zwa1 gene coding for the Zwa1 protein (DE 19959328.0; DSM 13115; EP-A-1111062);
the tpi gene coding for triose-phosphate isomerase (Eikmanns, J. Bacteriol. 174:6076-6086 (1992));
the pgk gene coding for 3-phosphoglycerate kinase (Eikmanns, J. Bacteriol. 174:6076-6086 (1992));
the dapA gene coding for dihydrodipicolinate synthase (EP-B 0 197 335).

It may also be advantageous for the production of amino acids, especially L-lysine, in addition to attenuating the gene coding for TipA, at the same time to attenuate, especially reduce the expression of, one or more genes selected from the group:
the ccpA1 gene coding for a catabolite control protein A (EP1311685);
the pck gene coding for phosphoenol pyruvate carboxykinase (DSM 13047, EP-A-1094111);
the pgi gene coding for glucose-6-phosphate isomerase (DSM 12969; EP-A-1087015; WO 01/07626);
the poxB gene coding for pyruvate oxidase (DSM 13114; EP-A-1096013);
the fda gene coding for fructose bisphosphate aldolase (Mol. Microbiol. 3 (11):1625-1637 (1989); Accession Number X17313); and
the zwa2 gene coding for the Zwa2 protein (DSM 13113; EP-A-1 106693).

Finally, it may be advantageous for the production of amino acids, in addition to attenuating the gene coding for TipA, to exclude undesirable secondary reactions (Nakayama: “Breeding of Amino Acid Producing Microorganisms,” in: Overproduction of Microbial Products, Krumphanzl, Sikyta, Vanek (eds.), Academic Press, London, UK, 1982).

The invention also includes microorganisms produced as described herein, which can be grown continuously or discontinuously during a batch process, a fed batch process or repeated fed batch process for the purpose of producing an L-amino acid. A summary of known cultivation methods is described in the textbook of Chmiel (Bioprozesstechnik 1. Einführung in die Bioverfahrenstechnik, Gustav Fischer Verlag, Stuttgart, 1991) and in the textbook of Storhas (Bioreaktoren und periphere Einrichtungen, Vieweg Verlag, Braunschweig/Wiesbaden, 1994).

The culture medium to be used must meet the requirements of the strains in question. Descriptions of culture media for various microorganisms are to be found in the handbook Manual of Methods for General Bacteriology of the American Society for Bacteriology (Washington D.C., USA, 1981). Carbon sources that may be used include: sugars and carbohydrates, such as glucose, sucrose, lactose, fructose, maltose, molasses, starch and cellulose, oils and fats, such as soybean oil, sunflower oil, groundnut oil and coconut oil, fatty acids, such as palmitic acid, stearic acid and linoleic acid, alcohols, such as glycerol and ethanol, and organic acids, such as acetic acid. These substances can be used individually or in the form of a mixture.

As a source of nitrogen organic nitrogen-containing compounds may be used, such as peptones, yeast extract, meat extract, malt extract, corn steep liquor, soybean flour and urea, or inorganic compounds, such as ammonium sulfate, ammonium chloride, ammonium phosphate, ammonium carbonate and ammonium nitrate. The nitrogen sources can be used individually or in the form of a mixture.

Phosphorus sources that may be used include: phosphoric acid, potassium dihydrogen phosphate or dipotassium hydrogen phosphate or the corresponding sodium-containing salts.

The culture medium must also contain salts of metals, such as, magnesium sulfate or iron sulfate, which are necessary for growth.

Finally, essential growth substances, such as amino acids and vitamins, may be used in addition to the above-mentioned substances. Suitable precursors may also be added to the culture medium. The mentioned substances may be added to the culture in the form of a single batch, or they may be fed in during the cultivation.

In order to control the pH of the culture, basic compounds, such as sodium hydroxide, potassium hydroxide, ammonia or ammonia water, or acidic compounds, such as phosphoric acid or sulfuric acid, may be used. In order to control the development of foam, anti-foams, such as fatty acid polyglycol esters, may be used. In order to maintain the stability of plasmids, substances having a selective action, such as antibiotics, may be added to the medium. In order to maintain aerobic conditions, oxygen or gas mixtures containing oxygen, such as air, are introduced into the culture.

The temperature of the culture is normally from 20° C. to 45° C. and preferably from 25° C. to 40° C.

The culture is continued until the maximum amount of the desired product has formed. This aim is normally achieved within a period of from 10 hours to 160 hours. Methods of determining L-amino acids are known in art and may be used in conjunction with the invention. The analysis may be carried out as described in Spackman, et al. (Anal. Chem. 30:1190 (1958)) by anion-exchange chromatography with subsequent ninhydrin derivatisation, or it may be carried out by reversed phase HPLC, as described in Lindroth et al. (Anal. Chem. 51:1167-1174 (1979)).

The following microorganism was deposited as a pure culture on 15 Feb. 2002 with the Deutsche Sammlung für Mikroorganismen und Zellkulturen (DSMZ, Braunschweig, Germany) in accordance with the Budapest Treaty: Escherichia coli Top10/pCR2.1tipAint as DSM 14816.

### BRIEF DESCRIPTION OF THE DRAWINGS

FIG. 1: The figure shows a map of plasmid pCR2.1tipAint. When indicating the number of base pairs, the values are approximate values obtained within the scope of the reproducibility of measurements.

### EXAMPLES

### Example 1

### Preparation of an Integration Vector for Integration Mutagenesis of the tipA Gene

Chromosmal DNA is isolated from strain ATCC 13032 by the method of Eikmanns et al. (Microbiol. 140:1817-1828 (1994)). On the basis of the known sequence of the tipA gene for C. glutamicum, the following oligonucleotides are selected for the polymerase chain reaction:

| | |
|---|---|
| tipA-int1: | |
| 5′CGC CTT TAC ACA GAA GAC G 3′ | (SEQ ID NO:1) |
| tipA-int2: | |
| 5′GTG TAC CAC TGA CCG ATG C 3′. | (SEQ ID NO:2) |

The primers shown are synthesised by MWG Biotech (Ebersberg, Germany), and the PCR reaction is carried out according to the standared PCR method of Innis, et al. (PCR Protocols, A Guide to Methods and Applications, Academic Press, 1990) using Taq polymerase from Boehringer Mannheim (Germany, product description Taq DNA polymerase, Product No. 1 146 165). With the aid of the polymerase chain reaction, the primers permit the amplification of an internal fragment of the tipA gene having a size of 482 bp. The product so amplified is investigated by electrophoresis in a 0.8% agarose gel.

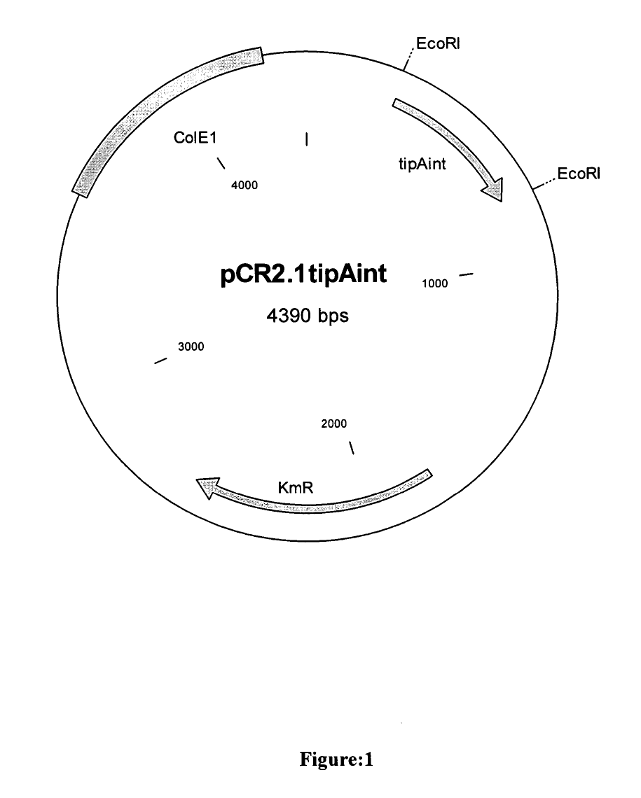

The amplified DNA fragment is ligated into vector pCR2.1-TOPO (Mead et al., Bio/Technology 9:657-663 (1991)) using the TOPO TA Cloning Kit from Invitrogen Corporation (Carlsbad, Calif., USA; Catalog Number K4500-01). E. coli strain TOP10 is then electroporated with the ligation batch (Hanahan, in: DNA Cloning. A practical approach, vol. 1, IRL-Press, Oxford, Washington D.C., USA, 1985). The selection of plasmid-carrying cells is carried out by plating out the transformation batch on LB agar (Sambrook et al., Molecular Cloning: A Laboratory Manual, 2^{nd} Ed., Cold Spring Harbor Laboratory Press, Cold Spring Harbor, N.Y., 1989) which has been supplemented with 50 mg/l kanamycin. Plasmid DNA is isolated from a transformant with the aid of the QIAprep Spin Miniprep Kit from Qiagen and is tested by restriction with the restriction enzyme EcoRI and subsequent agarose gel electrophoresis (0.8%). The plasmid is named pCR2.1tipAint and is shown in FIG. 1. A microorganism carrying this plasmid, Escherichia coli Top10/pCR2.1tipAint, is deposited as pure culture DSM 14816 on 15 Feb. 2002 with the Deutsche Sammlung für Mikroorganismen und Zellkulturen (DSMZ, Braunschweig, Germany) in accordance with the Budapest Treaty.

### Example 2

### Integration Mutagenesis of the tipA Gene in the Strain DSM 5715

Vector pCR2.1tipAint described in Example 1 is electroporated into Corynebacterium glutamicum DSM 5715 by the electroporation method of Tauch et al. (FEMS Microbiol. Lett. 123:343-347 (1994)). Strain DSM 5715 is an AEC-resistant lysine producer, and is described in EP-B-0435132. Vector pCR2.1tipAint is unable to replicate independently in DSM5715 and is retained in the cell only if it has integrated into the chromosome of DSM 5715. The selection of clones with pCR2.1tipAint integrated into the chromosome is effected by plating out the electroporation batch on LB agar (Sambrook et al., Molecular Cloning: A Laboratory Manual, 2^{nd} ed., Cold Spring Harbor Laboratory Press, Cold Spring Harbor, N.Y.) which has been supplemented with 15 mg/l kanamycin. A selected kanamycin-resistant clone which has the plasmid pCR2.1tipAint inserted within the chromosomal tipA gene of DSM5715 was designated DSM5715::pCR2.1tipAint.

### Example 3

### Production of Lysine

The C. glutamicum strain DSM5715::pCR2.1tipAint obtained in Example 2 is cultivated in a nutrient medium suitable for the production of lysine, and the lysine content in the culture supernatant is determined. To that end, the strain is first incubated for 24 hours at 33° C. on an agar plate with an appropriate antibiotic (brain-heart agar with kanamycin at 25 mg/l). Starting from this agar plate culture, a pre-culture is inoculated (10 ml of medium in a 100 ml Erlenmeyer flask). CgIII complete medium is used as the medium for the pre-culture.

### Cg III Medium

| | |
|---|---|
| NaCl | 2.5 g/l |
| Bacto-peptone | 10 g/l |
| Bacto-yeast extract | 10 g/l |
| Glucose (autoclaved separately) | 2% (w/v) |

The pH value is adjusted to pH 7.4

Kanamycin (25 mg/l) is added thereto. The pre-culture is incubated for 16 hours on a shaker at 33° C. and 240 rpm. A main culture is inoculated from this pre-culture, so that the initial OD (660 nm) of the main culture is 0.1 OD. MM medium is used for the main culture.

| | |
|---|---|
| CSL (corn steep liquor) | 5 g/l |
| MOPS (morpholinopropanesulfonic acid) | 20 g/l |
| Glucose (autoclaved separately) | 50 g/l |
| Salts: | |
| (NH₄)₂SO₄) | 25 g/l |
| KH₂PO₄ | 0.1 g/l |
| MgSO₄ * 7 H₂O | 1.0 g/l |
| CaCl₂ * 2 H₂O | 10 mg/l |
| FeSO₄ * 7 H₂O | 10 mg/l |
| MnSO₄ * H₂O | 5.0 mg/l |
| Biotin (sterilised by filtration) | 0.3 mg/l |
| Thiamin * HCl (sterilised by filtration) | 0.2 mg/l |
| Leucine (sterilised by filtration) | 0.1 g/l |
| CaCO₃ | 25 g/l |

CSL, MOPS and the salt solution are adjusted to pH 7 with ammonia water and autoclaved. The sterile substrate and vitamin solutions are then added, as well as the dry autoclaved CaCO₃. Cultivation is carried out in a volume of 10 ml in a 100 ml Erlenmeyer flask with baffles. Kanamycin (25 mg/l) is added. Cultivation is carried out at 33° C. and 80% humidity.

After 72 hours, the OD is determined at a measuring wavelength of 660 nm using a Biomek 1000 (Beckmann Instruments GmbH, Munich). The amount of lysine that has formed is determined using an amino acid analyser from Eppendorf-BioTronik (Hamburg, Germany) by ion-exchange chromatography and post-column derivatisation with ninhydrin detection. The result of the test is shown in Table 1.

TABLE 1

| | OD | Lysine HCl |
|---|---|---|
| Strain | (660 nm) | g/l |
| DSM5715 | 8.2 | 13.6 |
| DSM5715::pCR2.1tipAint | 10.5 | 15.1 |

### Abbreviations

The abbreviations and names used have the following meanings:

| | |
|---|---|
| KmR: | kanamycin resistance gene |
| EcoRI: | cleavage site of the restriction enzyme EcoRI |
| tipAint: | internal fragment of the tipA gene |
| ColE1: | origin of replication of plasmid ColE1 |

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1. A process for producing an L-amino acid product, comprising:
a) fermenting a coryneform bacterium producing said L-amino acid in a fermentation medium, wherein the the transcription regulator TipA has been attenuated in said bacterium;
b) allowing the concentration of said L-amino acid to increase either in said fermentation medium or in said bacterium; and
c) collecting said L-amino acid from either said fermentation medium or said bacterium to produce said amino acid product.

2. The process of claim 1, wherein attenuation of TipA is the result of the disruption of the tipA gene by homologous recombination.

3. The process of claim 1, wherein said L-amino is L-lysine.

4. The process of claim 1, wherein said L-amino acid product further comprises biomass and other constituents from said fermentatiom medium.

5. The process of claim 1, wherein at least one gene in the biosynthesis pathway of said L-amino acid is overexpressed in said bacterium.

6. The process of claim 1, wherein said L-amino acid is L-lysine, and said bacterium overexpresses one or more genes selected from the group consisting of:
a) the lysC gene coding for a feedback-resistant aspartate kinase;
b) the lysE gene coding for lysine export;
c) the gap gene coding for glyceraldehyde-3-phosphate dehydrogenase;
d) the pyc gene coding for pyruvate carboxylase;
e) the zwf gene coding for glucose-6-phosphate dehydrogenase;
f) the mqo gene coding for malate:quinone oxidoreductase;
g) the zwa1 gene coding for the Zwa1 protein;
h) the tpi gene coding for triose-phosphate isomerase;
i) the pgk gene coding for 3-phosphoglycerate kinase; and
j) the dapA gene coding for dihydrodipicolinate synthase.

7. The process of claim 1, wherein at least one gene in a metabolic pathway that reduces the formation of the desired L-amino acid is at least partially excluded.

8. The process of claim 1, wherein said L-amino acid is L-lysine and at least one gene is attenuated, said at least one gene being selected from the group consisting of:
a) the ccpA1 gene coding for a catabolite control protein A;
b) the pck gene coding for phosphoenolpyruvate carboxykinase;
c) the pgi gene coding for glucose-6-phosphate isomerase;
d) the poxB gene coding for pyruvate oxidase;
e) the fda gene coding for fructose bisphosphate aldolase; and
f) the zwa2 gene coding for the Zwa2 protein.

9. The process of claim 1 wherein said bacterium is of the species Corynebacterium glutamicum.

10. A process for producing an L-lysine product, comprising:
a) fermenting a coryneform bacterium producing said L-lysine in a fermentation medium, wherein the gene coding for the transcription regulator TipA has been disrupted by homologous recombination in said bacterium;
b) allowing the concentration of said L-lysine to increase either in said fermentation medium or in said bacterium; and
c) collecting said L-lysine from either said fermentation medium or said bacterium to produce said L-lysine product.

11. The process of claim 10, wherein said L-lysine product further comprises biomass and other constituents from said fermentatiom medium.

12. The process of claim 10, wherein said bacterium overexpresses one or more genes selected from the group consisting of:
a) the lysC gene coding for a feedback-resistant aspartate kinase;
b) the lysE gene coding for lysine export;
c) the gap gene coding for glyceraldehyde-3-phosphate dehydrogenase;
d) the pyc gene coding for pyruvate carboxylase;
e) the zwf gene coding for glucose-6-phosphate dehydrogenase;
f) the mqo gene coding for malate:quinone oxidoreductase;
g) the zwa1 gene coding for the Zwa1 protein;
h) the tpi gene coding for triose-phosphate isomerase;
i) the pgk gene coding for 3-phosphoglycerate kinase; and
j) the dapA gene coding for dihydrodipicolinate synthase.

13. The process of claim 10, wherein at least one gene is attenuated in said bacterium, said at least one gene being selected from the group consisting of:
a) the ccpA1 gene coding for a catabolite control protein A;
b) the pck gene coding for phosphoenolpyruvate carboxykinase;
c) the pgi gene coding for glucose-6-phosphate isomerase;
d) the poxB gene coding for pyruvate oxidase;
e) the fda gene coding for fructose bisphosphate aldolase; and
f) the zwa2 gene coding for the Zwa2 protein.

14. The process of claim 10, said bacterium is of the species Corynebacterium glutamicum.

15. A coryneform bacterium in which the gene coding for the transcription regulator TipA has been attenuated.

16. The coryneform bacterium of claim 15, wherein said gene cosing for TipA has been disrupted by homologous recombination.

17. The coryneform bacterium of claim 16, wherein said bacterium overexpresses one or more genes selected from the group consisting of:
a) the lysC gene coding for a feedback-resistant aspartate kinase;
b) the lyse gene coding for lysine export;
c) the gap gene coding for glyceraldehyde-3-phosphate dehydrogenase;
d) the pyc gene coding for pyruvate carboxylase;
e) the zwf gene coding for glucose-6-phosphate dehydrogenase;
f) the mqo gene coding for malate:quinone oxidoreductase;
g) the zwa1 gene coding for the Zwa1 protein;
h) the tpi gene coding for triose-phosphate isomerase;
i) the pgk gene coding for 3-phosphoglycerate kinase; and
j) the dapA gene coding for dihydrodipicolinate synthase.

18. The coryneform bacterium of claim 10, wherein at least one gene is attenuated, said at least one gene being selected from the group consisting of:
a) the ccpA1 gene coding for a catabolite control protein A;
b) the pck gene coding for phosphoenolpyruvate carboxykinase;
c) the pgi gene coding for glucose-6-phosphate isomerase;
d) the poxB gene coding for pyruvate oxidase;
e) the fda gene coding for fructose bisphosphate aldolase; and
f) the zwa2 gene coding for the Zwa2 protein.

19. The coryneform bacterium of claim 18, wherein said at least one gene is attenuated due to its being disrupted by homologous recombination.

20. The coryneform bacterium of claim 10, wherein said bacterium is of the species Corynebacterium glutamicum.
